(19) United States
(12) Patent Application Publication
(10) Pub. No.: US 2011/0142907 A1
(43) Pub. Date: Jun. 16, 2011

(54) Polymer for tissue bonding

(76) Inventors: Kevin S. Marchitto, Golden, CO (US); Stephen T. Flock, Arvada, CO (US); Amy Benedict, Aurora, CO (US)

(21) Appl. No.: 12/927,938

(22) Filed: Nov. 30, 2010

Related U.S. Application Data
(60) Provisional application No. 61/265,239, filed on Nov. 30, 2009.

(51) Int. Cl. A61F 2/00; A61K 9/70; A61K 38/38; A61K 38/39; A61K 38/36; A61P 17/02; H01B 1/12

(52) U.S. Cl. 424/426; 424/444; 514/16.5; 514/13.6; 252/500

(57) ABSTRACT

Provided herein are polymeric adhesives which may be cured by inductive heating and which are suitable for treating tissue in an individual to effect a weld between one or more tissues or between a tissue and at least one non-tissue substrate. Also provided are methods using the adhesives and methods of manufacture.

### CROSS-REFERENCE TO RELATED APPLICATION

This nonprovisional application claims benefit of priority under 35 U.S.C. §119(e) of provisional applications U.S. Ser. No. 61/265,239, filed Nov. 30, 2009, now abandoned, the entirety of which are hereby incorporated by reference.

### BACKGROUND OF THE INVENTION

1. Field of Invention

The present invention relates generally to the fields of biomedical engineering, materials engineering, polymer biochemistry and surgery. More specifically, the present invention provides a biomaterial adhesive which may be a polymer and methods and devices for activating the biomaterial in order to improve the ease with which tissue can be fused to tissue or to other materials, or with which cavities in tissues can be sealed.

2. Description of the Related Art

Conventional methods of wound closure following surgery consist of applying sutures or staples to join two or more tissues that have been dissected. While these methods are generally successful, at times complications arise due to inadequate closure of the wound that could result in the tissues separating or in “leakiness”. In particular, the quality of suturing depends on manual dexterity of the surgeon and adequate access to the wound. Current designs of surgical clips can slip if applied incorrectly or accidentally disturbed. Surgical clips can also cause damage to the vessels or structures to which they are applied if the surgeon applies excessive compression force. With the increasing use of minimally invasive surgical methods, such as endoscopy, wound access and the efficient closure of wounds has become a significant issue in medicine.

The most common alternative methods to the traditional mechanical means of closing incisions, wounds, and anastomoses include use of cyanoacrylate adhesives and fibrin sealants. More recently, wound sealing approaches, which employ methods of directing energy to the tissue which as a consequence adheres to proximal tissue, have been tested and used clinically. Commercial electrosurgery and electrocautery devices commonly are used for sealing internal wounds, such as those arising through surgical intervention. Inventions for sealing vessels using other forms of electromagnetic energy have been published. U.S. Pat. No. 6,033,401 describes a device to deliver adhesive and apply microwave energy to effect sealing of a vessel. U.S. Pat. No. 6,179,834 discloses a vascular sealing device to provide a clamping force, while radiofrequency energy is applied, until a particular temperature or impedance is reached. U.S. Pat. No. 6,132,429 describes using a radiofrequency device to weld blood vessels closed and monitoring the process by changes in tissue temperature or impedance. Nevertheless, these devices are generally unsuitable for the purpose of occluding a wound thereby enhancing long-term healing.

Over the past fifteen years, a significant amount of scientific research has focused on using laser heated “solder” for “welding” tissues such as blood vessels (1-2). Research has been done on laser tissue welding with albumin solders which are an improvement over conventional suture closure because it offers an immediate watertight tissue closure, decreased operative time, especially in microsurgical or laparoscopic applications, reduced trauma, and elimination of foreign body reaction to sutures, collagen-based plugs and clips. The procedure has been enhanced with the use of advanced solders, strengthening structures, concurrent cooling, and added growth factors as disclosed, for example, in U.S. Pat. No. 6,221,068.

Use of lasers for tissue welding appeared very promising, however, the techniques presents certain limitations. The laser energy must be manually directed by the surgeon which leads to operator variability. Additionally, the radiant energy is not dispersed evenly throughout the tissue. The high energy at the focal point may result in local burns and the heating effect drops off rapidly at a small distance from the focal point. Finally, lasers are expensive and cannot be miniaturized easily.

A number of patents describe using electromagnetic energy, often in the form of laser or other radiant energy, to heat tissue or a biocompatible “solder” to effect tissue sealing or fusion. U.S. Patent Publication Nos. 2003/019862 and 2003/0195499, for example, describe microwave antennae suitable for cutting or ablating tissue. U.S. Pat. No. 5,925,078 describes using a form of energy, such as microwaves or radiofrequency, to fuse endogenous collagen fibrils in tissue, whereupon the strength of the fusion is enhanced by subsequent chemically-induced protein cross-linking. U.S. Pat. No. 6,669,694 uses a different application of energy, in the form of a vaporized biocompatible material, which exits an applicator to impinge on tissue in order to effect a beneficial tissue effect. Neither Anderson nor Shadduck describe using an additional adhesive during the described processes.

Menovsky and co-workers (Effect of CO.sub.2-milliwatt laser on peripheral nerves: Part II. A histological and functional study, Microsurgery 20, pp 150-155, 2000) showed that by using an albumin solder applied to a sciatic nerve and cured with the radiant energy produced by a CO2 laser, it was possible to elicit nerve repair without causing unacceptable thermal side-effects. Lauto et al. (Laser-activated solid protein bands for peripheral nerve repair: an vivo study. Lasers in Surgery & Medicine. 21, pp 134-41, 1997) and McNally-Heintzelman et al. (Scaffold-enhanced albumin and n-butyl-cyanoacrylate adhesives for tissue repair: ex vivo evaluation in a porcine model. Biomedical Sciences Instrumentation. 39, pp 312-7, 2003) found beneficial results of laser-nerve welding using other laser radiant energy and differing adhesive compositions. Nevertheless, the lack of control and the inability to induce uniform heating in the nerve as a result of laser irradiation restricts the utility of laser-nerve welding to the laboratory. Because of this, the procedure is not used in the clinic on human patients.

Biomolecules such as fibrin, elastin, and albumin have been or are used to “glue” tissue to tissue. A number of patents describe the “activation” of these biomolecules to form “welds” through irradiation, often in the form of laser radiant energy, but sometimes in the form of ultrasound or radiofrequency waves. The applied energy is believed to denature the molecules, which then adhere to one another or cross-link to one-another and to protein in tissues, thereby effecting a union between the tissues.

Tissue solders used in conjunction with laser tissue welding are biocompatible, do not resorb quickly, and may be effective in wet environments. They do, however, present other problems, e.g., uneven welding due to high heat concentrations, rigid and brittle closure sites, again limiting future mechanics, and thermal damage to surrounding tissue. The cost of laser operation is a particular drawback. The use of lasers does offer direct crosslinking of adhesive to tissue.

The common challenges in tissue closure, sealing, and healing, is creating a closure site that will immediately seal, endure mechanical stresses and return the tissue to its natural productivity nearly immediately. An important favor behind biological glues is biocompatibility and tissue crosslinking; however, the glues routinely under perform and limit the tissues return to status quo. A new concept in tissue adhesives is polymer biomaterials and hydrogels, for their tissue like resemblance and behavior. Additionally, a hydrogel type adhesive can be embedded with nutrients, proteins and sugars similar to the extracellular matrix—making the adhesive an extension of the tissue. Polymers and particularly hydrogels have gained momentum for biomedical applications because of their tissue likeness, biocompatibility and alterability for different applications: tissue regeneration (U.S. Pat. No. 6,156,572), in situ bonds (U.S. Pat. No. 6,524,327), oral bandages (U.S. Pat. No. 6,492,573), invertebrate discs (U.S. Pat. No. 6,893,466), wound dressing (U.S. Pat. Nos. 5,480,717, 5,112,618, 5,429,589, 5,674,523, 6,180,132), orthopedic implants (U.S. Pat. No. 7,008,635), corneal implants (U.S. Pat. No. 6,884,261) etc. Numerous polymers have been explored for hydrogel formation and implantation, including but not limited to: polyethylene glycol, polyvinyl alcohol, polylactic acid, polyglycolic acid, poly lactic-co glycolic acid, polyvinyl chloride, poly(vinylpyrro1idone), polyacrylamide, methacrylates; Polysacchrides including cellulose, specifically, hydroxylpropyl cellulose and hydroxymethyl propyl cellulose, aliginates, carageenans (lambda, iota, kappa), pullulan; Glycosaminoglycans such as hyaluronic acid. All of these have different innate properties as well as methods to polymerization and/or crosslinking allowing for water sorption and tissue like behaviors. The most notable crosslinking methods include chemical, thermal, photo. Additionally, lypholization can induce crosslinking. In this method, crosslinking occurs from the concentration of the polymer chains creating a Van der Waals attraction between sidegroups.

Differing crosslinking mechanisms controls formation of resultant polymer. Meaning, the use of hydrogels and biopolymers allows the user to have more control of the end polymer: matrixes, laminates, multiple layers, layers with different functions and mechanical properties, shaping etc. are all controllable possibilities with the numerous crosslinking methods. This is an added benefit to the use of hydrogels as one can controllably mimic innate tissues through the use of crosslinking.

Despite the growing understanding and benefits of biopolymers and hydrogels for numerous in vivo applications there lacks methodology for utilizing the hydrogel as an adhesive, which requires that the hydrogel adhere and seal the tissue upon contact, in essence, crosslink with the tissue. This art has a two fold requirement: embedding the hydrogel with biomolecules that serve as the “fusion composition” such as collagen, fibrin, elastin, and/or albumin, whereby the molecules are denatured by an applied energy which then renatures or crosslinks effecting a bond, and secondly a method of activating such molecules within the hydrogel. In laser tissue welding, heat adjuvantly crosslinks the biocompatible solder to the tissue; however, the drawbacks of laser tissue welding repeated as supra: brittle closure sites, high cost, high heat concentrations. A non-contact heat source used in conjunction with novel hydrogel tissue adhesives could dynamically transform tissue sealing and tissue adhesives.

Commercial electrosurgery and electrocautery devices commonly are used for sealing internal wounds, such as those arising through surgical intervention. Inventions for sealing vessels using other forms of electromagnetic energy have been published. U.S. Pat. No. 6,033,401 describes a device to deliver adhesive and apply microwave energy to effect sealing of a vessel. U.S. Pat. No. 6,179,834 discloses a vascular sealing device to provide a clamping force while radiofrequency energy is applied until a particular temperature or impedance is reached. U.S. Pat. No. 6,132,429 describes using a radiofrequency device to weld blood vessels closed and monitoring the process by changes in tissue temperature or impedance. Nevertheless, these devices are generally unsuitable for the purpose of occluding a wound thereby enhancing long-term healing.

U.S. Pat. No. 5,669,934 describes a method for joining or restructuring tissue consisting of providing a preformed film or sheet of a collagen and/or gelatin material which fuses to tissue upon the application of continuous inert gas beam radiofrequency energy. Similarly, U.S. Pat. No. 5,569,239 describes laying down a layer of energy reactive adhesive material along the incision and closing the incision by applying energy, either optical or radiofrequency energy, to the adhesive and surrounding tissue. Similarly U.S. Pat. Nos. 5,209,776 and 5,292,362 describe a tissue adhesive that is principally intended to be used in conjunction with laser radiant energy to weld severed tissues and/or prosthetic material together. U.S. Pat. No. 6.110.212 describes the use of elastin and elastin-based materials which are biocompatible and can be used to effect anastomoses and tissue structure sealing upon the application of laser radiant energy. The stated benefits, inter alia, are the biocompatible and ubiquitous nature of elastin.

U.S. Pat. No. 6,302,898 describes a device to deliver a sealant and energy to effect tissue closure. It also discloses pre-treating the tissue with energy in order to make the subsequently applied sealant adhere better. PCT Appplication No. WO 99165536 describes tissue repair by pretreating the substantially solid biomolecular solder prior to use. U.S. Pat. No. 5,713,891 discloses the addition of bioactive compounds to the tissue solder in order to enhance the weld strength or to reduce post-procedure hemorrhage.

U.S. Pat. No. 6,221,068 teaches the importance of minimizing thermal damage to the tissue to be welded. The method employs pulsed laser irradiation and allowing the tissue to cool to nearly the initial temperature between each heating cycle. U.S. Pat. No. 6,323,037 describes the addition of an “energy converter” to the solder mixture such that optical energy will be efficiency and preferentially absorbed by the solder which subsequently will effect a tissue weld.

Inductive heating is a non-contact process whereby electrical currents are induced in electrically conductive materials (susceptors) by a time-varying magnetic field. Generally, induction heating is an industrial process often used to weld, harden or braze metal-containing parts in manufacturing where control over the heating process and minimized contact with the workpiece are critical. Basically, radiofrequency power is coupled to a conducting element, such as a coil of wire, which serves to set up a magnetic field of a particular magnitude and spatial extent. The induced currents or Eddy currents flow in the conductive materials in a layer referred to as the skin depth d), given by: d=√(2ρ/μω) where ω is frequency (rads/s), ρ is resistivity (ohm-m) and μ is the permeability (Webers/amp/m) which is the product of po the permeability of free space and pr the relative permeability of the material.

The magnetic permeability of a material is quantification of the degree to which it can concentrate magnetic field lines. Note, however, that the permeability is not constant in ferromagnetic substances like iron, but depends on the magnetic flux and temperature. The skin depth at room temperature at 1 MHz electromagnetic radiation in copper is 0.066 mm and in 99.9% iron is 0.016 mm.

The consequence of current flowing is Joule, or I′R, heating. The skin-depth formula leads to the conclusion that, with increased frequency, the skin depth becomes smaller. Thus, higher frequencies favor efficient and uniform heating of smaller components. In certain situations localized heat can also be generated through hysteresis losses or frictional heating, referred to as dielectric hysteresis heating in non-conductors, as the susceptor moves against physical resistance in the surrounding material. Consideration of Joule heating alone results in a formula for the power-density P (W/cm³) in the inductively-heated material:

P=4π H² μₒ μᵣ f M,

where H is the root means square (RMS) magnetic field intensity (A/m), f is frequency (Hz), M is a power density transmission factor (unitless) which depends on the physical shape of the heated material and skin depth and diameter of the part to be heated (4-5).

M, which is equal to the product of F and d/δ, where F is a transmission factor and d is the diameter of the part, can be shown to be maximally about 0.2 when the object diameter is 3.5 times the skin depth, and when certain other assumptions are made.

Thus, for a given frequency there is a diameter for which the power density is a maximum; or equivalent, there is a maximum frequency for heating a part of a certain diameter below which heating efficiency drops dramatically and above which little or no improvement of heating efficiency occurs. It can also be shown that the power density of inductively heated spheres is much higher than solid spheres of the same material.

Conventional applications of induction heating involve welding, hardening, brazing or forging metal components. Some applications have been reported which use the process to cure adhesives in bonding processes or for applying coatings. U.S. Pat. No. 6,348,679 discloses compositions used in bonding two or more conventional materials where the interposed composition consists of a carrier and a susceptor, which may be at least in part composed of certain proteins. However the applications apply to conventional substrates such as films, metal substrates or wood.

There are only a few examples of the use of inductive heating in medical literature or for applications with biological materials. Principles of inductive heating have been applied to hyperthermia of cancer whereby large metallic “seeds” are inductively heated using a coil external to the body (6-7). Additionally, a recent report described the use of induction heating to heat nanocrystals coupled to DNA to locally denature DNA for the purpose of hybridization (8).

U.S. patent application Ser. No. 200210183829 describes inductively heating stents made of alloys with a high magnetic permeability and low Curie temperature for the purpose of destroying smooth muscle cells in restenosing blood vessels.

Common problems exist throughout the prior art. These include tissue damage due to uneven heating, unknown and/or uncontrollable thermal history, i.e., time temperature profile, and relatively high cost. It is notable that a consistent means of treatment and control are desirable. The Code of Federal Regulations, 21 CFR 860.7(e)(I), establishes that there is “reasonable assurance that a device is effective when it can be determined, based upon valid scientific evidence, that in a significant portion of the target population, the use of the device will provide clinically significant results.” Devices that cannot be shown to provide consistent results between patients, or even within a patient upon multiple use, will have minimal utility and may not be approved, if approved, for broad use. Beyond devices, it is generally desirable to develop medical products with critical controls that can deliver precise results.

A tissue fusion wound closure system: device and biopolymer/hydrogel adhesive that overcomes the many deficiencies described in the prior art would improve patient care and reduce costs while supporting the expanded use of minimally invasive surgery. The inventors have recognized an increased need for a closure device and method that maintains the clinical advantages of laser-tissue welding, but eliminates the limitations. The prior art is deficient in devices and methods for minimally-invasive methods that use electromagnetic energy to controllably alter a biopolymer through molecular alterations and/or mechanical shrinkage to adhere to tissue. And for such adhesives there has yet to one that mimics innate tissue: on that maintains integrity, flexibility and biomechanics during the would healing process. The present invention fulfills this longstanding need and desire in the art.

### SUMMARY OF THE INVENTION

The present invention is directed to an adhesive composition comprised of one or more layers of polymer, each of which may be comprised of a substrate and a conductive element.

The present invention also is directed to an adhesive composition comprised of one or more layers of a biopolymer or a hydrogel, each of which may be comprised of a substrate and a conductive element.

### BRIEF DESCRIPTION OF THE DRAWINGS

FIGS. 1A-1B show Franz diffusion chamber disassembled (FIG. 1A) and with stapled, sealed lung tissue in place (FIG. 1B).

FIG. 2 shows a graph depicting that the biofusionary adhesive beginning to leak at 68 mmHg before bursting at 85 mmHg. Average static lung pressure is 22 mmHg.

FIG. 3 shows a filamentous adhesive formulation used to seal catheter sites.

FIG. 4 shows a graph depicting that biofusionary adhesive used with coil cured within 7 seconds and showed great consistency between samples. The results supports minimally invasive procedures that seal over the open wounds within seconds.

FIG. 5 shows a graph depicting that the seal showed consistent resistance to pressure with increasing pressure indicating a strong uniform seal had formed within the tissue.

FIG. 6 shows a graph depicting the degree of swelling of the bilayer polymer formulation. The formulation reached 200% within 5 minutes indicating loose chain entanglement rather than cross-linking (n=6).

FIGS. 7A-7B show scanning electron microscope images which reveal that the different morphologies of the opposing bilayer components and also porosity allowing the tissue ingrowth. FIG. 7A is PVA and salt layer while FIG. 7B is PVA and albumin layer. The pores on the protein side appear to 10-50 mm.

FIGS. 8A-8B show images of stapled and sealed lung tissue from rabbit 1 (100× orig. mag. FIG. 8A) and rabbit 2 (20× orig. mag. FIG. 8B).

FIG. 9 shows emulsion, freeze-spray and pulverized micro-particle production methods produced particles of similar size.

### DETAILED DESCRIPTION OF THE INVENTION

The present invention relates to an adhesive composition comprising one or more layers of polymer, each of which may comprise a substrate and a conductive element. Representative conductive elements include but are not limited to an ionic salt, conductive polymers, transition metal, ionic solution, circuit pattern, or ferromagnetic material. The substrate may, for example, be cross-linked, welded, or fused to the tissue upon heat activation. The cross-linking may be achieved through chemical, thermal, lyophilization, freeze-thaw, or photopolymerization methods. The heat activation may be accomplished by any method known to one of ordinary skill in this art including but not limited to by inductive coupling or capacitive coupling. Representative polymers include polyethylene glycol, polyvinyl alcohol, polylactic acid, polyglycolic acid, polycaprolactone, poly lactic-co glycolic acid, polyvinyl chloride, poly(vinylpyrro1idone), polyacrylamide, methacrylates, polyethylene oxide, polysaccharide, or glycosaminoglycan. Representative polysaccharides include cellulose, hydroxymethyl propyl cellulose, aliginates, carageenans, or pullulan. A representative glycosaminoglycan is hyaluoronic acid. The polymer may be solid. The polymer may also contain indentations or holes that allow tissues to grow into the polymer. The polymer may be casted, molded, or extruded to a specific three-dimensional geometric shape. Representative geometric shapes include rectangles, tubes, cylinders, or cones.

The present invention also relates to an adhesive composition comprised of one or more layers of a biopolymer or a hydrogel, each of which may be comprised of a substrate and a conductive element. The biopolymer may be albumin, collagen, fibrin, or elastin. The elastin, albumin, or collagen may be present at concentrations of about 1-75%. The concentrations may be further narrowed to about 1-20%. The hydrogel may further contain albumin, collagen, elastin, silk, lignin, dextran, soy derivatives, or polyglutamic acid. The hydrogel may further contain a pharmaceutical, anti-coagulant, antithrombotic, antibiotic, hormone, anti-inflammatory agent, anti-viral agent, or anti-fungal agent. The surface of the hydrogel may be functionalized or altered. Representative conductive elements include ionic salts, conductive polymers, transition metals, ionic solutions, circuit patterns, or ferromagnetic materials. The conductive element may be separate but proximal to the hydrogel. The polymer may be substituted with a bifunctional or heterofunctional cross-linker. The bifunctional cross-linkers may be chains of polyethylene glycol, or polyethylene oxide substituted with end units such as hydroxyls, maleimids, sulfhydryls, esters, or amines.

The present invention also relates to method of filling defects in tissue or attaching tissue together by applying the polymer described above and exposing the polymer to high frequency magnetic field.

The composition is an adhesive fusion composition comprised of a polymer, a conductive medium and cross-linking substrate. In the presence of a high frequency alternating magnetic field, the formation of eddy currents, or hysteresis, in the conductive medium leads to the generation of heat which in turn enables the substrate to cross-link, weld or fuse to tissue. The composition may be placed between layers of tissue in order to bind them together, between layers for tissue anastomosis, to fill defects in tissues, to expand tissues, to seal leaks in tissues, or between a tissue and a dressing that are to be welded or fused. For example, during wound closure, a dressing or other fastener containing such composition may be applied to the wound site and cured in place. Preferably, the composition is comprised of a polymer, preferably a biopolymer or hydrogel, which forms a network for conductive material and a substrate. In this embodiment the composition is thermally activated and acts as an adhesive.

More specifically, the polymer is self-supporting and three dimensional: “self-supporting” means that the material is solid and has adequate mechanical integrity to maintain its shape whereas “three dimensional” means that the material is one which is molded, extruded, or cast to maintain a specific geometric shape. The three dimensional structures may be relatively simple: rectangles, tubes, cylinders, cones etc. Alternatively the polymer may contain indentions or holes that allow tissue to grow into the polymer.

The substrate is a cross-linking element within the polymer and is typically a protein, for example albumin, collagen, fibrin or elastin. The protein component may be embedded within the polymer matrix or may be absorbed into the surface of the polymer.

In some embodiments, the polymer itself may be modified to act as substrate, particularly where the modification enables the polymer itself to cross-link directly to a substrate or tissue, for example by modification with a bifunctional or hetrofunctional crosslinker. These crosslinkers may be heterofunctional and bifunctional chains of polyethylene glycol and/or polyethylene oxide substituted with end functional units that may be hydroxyls, maleimides, sulfhydryls, esters, or amines. Said chains may also function as branching units that may be crosslinked by the similar means.

The conductive element within the composition may be a ferromagnetic material, an ionic solution, or other conductive medium. Examples of conductive medium include ionic salts, conductive polymers, and transition metals. Additionally, the conductive material may be separate but proximal to the hydrogel.

In a preferred embodiment, the composition is comprised of one or more layers of polymer, each of which in turn may be comprised of a cross-linking substrate and a conductive element.

Preferably, the materials that comprise the composition are able to be heated by inductive coupling, or optionally by capacitive coupling, and to produce a fusion in biomaterials. Said polymer may be a hydrogel or other synthetic material or biomaterial, or a naturally occurring material used to seal, cover, bind or replace part of a living system. The polymers are additionally biocompatible and may be comprised of polyethylene glycol, polyvinyl alcohol, polylactic acid, polyglycolic acid, polycaprolactone, poly lactic-co glycolic acid, polyvinyl chloride, poly(vinylpyrro1idone), polyacrylamide, methacrylates, polyethylene oxide; Polysacchrides including cellulose, specifically, hydroxylpropyl cellulose and hydroxymethyl propyl cellulose, aliginates, carageenans (lambda, iota, kappa), pullulan; Glycosaminoglycans such as hyaluronic acid. Each of these materials has unique properties as well as methods to polymerization and/or crosslinking which give rise to variable water sorption and tissue like behaviors. Thus, materials may be selected based on desirable characteristics such as tissue mimicry, or hydration.

Independent layers of a polymer may be created through chemical-, thermal-, and photopolymerization or crosslinking methods. Additionally, lyophilization and freeze-thaw methods can be used to promote crosslinking where Van der Waals attractions lead to close associations of chemical side chains, which may further lead to ionic or covalent linkages.

Chemical crosslinking makes use of available side groups on said polymer. The introduced chemical can induce substitutive crosslinking where side groups are substituted and then linked between each other. The chemical can also cause attractive grouping: where the sidegroups have an attraction to the introduced chemical causing the polymer to concentrate and thus crosslink. Chemicals used depend on the polymer at hand but may include sodium bicarbonate, calcium chloride, magnesium chloride, sodium chloride, ethylene glycol, polyethylene glycol, polyethylene oxide, divinyl ether and different concentrations thereof.

Alternatively, conductive materials may be incorporated to form electrically conductive patterns in polymers or on cloth substrates. Typical patterns can include circuit structures such as RF transmission lines, antennas, filters, and other conductive patterns equivalent to those of conventional printed circuits.

In a further embodiment, substrates comprised of proteins may be sensitive to cross-linking methods where radiation or chemicals may result in degradation or undesirable protein modification. For example, thermal crosslinking makes use of heat, typically 60-80° C., to initiate sidegroup attractions, which can result in denaturation of certain protein substrates. Ultraviolet light is used to crosslink other polymer formulations, however many proteins are sensitive to UV light, leading to degradation or aggregation. Therefore, it is an object of the invention to produce a polymer lattice whereby substrates are protected. For example, at least one layer is comprised of a polymer and substrate, while one or more different layers are comprised of conductive element and polymer, whereby the different layers are juxtaposed such that the generation of heat in one layer results in thermal effects in the layer or layers proximal to it.

The present invention generally relates to a device and method for heating a liquid, solid or gel fusion composition to be utilized as a means of heating biomolecules, particularly those in living systems. The device may consist of a source of electrical energy coupled to at least one electrode or a source of radiofrequency (RF) energy coupled to an applicator or induction coil to generate an electromagnetic field. Electrical energy or the oscillating magnetic field interacts with the fusion composition resulting in the production of heat substantially within the fusion composition.

As used herein, the term “weld” or “solder” may be used interchangeably to represent bonding, fusing or attaching of one or more substrates including sections of tissue to another section of tissue, to a dressing, or to a fastening device such as a clip, pin or staple.

Yet another embodiment of the present invention provides a method of treating tissue in an individual to effect a weld between a tissue and a substrate, comprising the steps of placing the device disclosed supra on the tissue of said individual; inductively heating the fusion composition comprising the device; and monitoring the device to control the extent of the weld between said tissue and said substrate(s). The steps of inductively heating the fusion composition and monitoring the weld process may be repeated at least once.

A preferred embodiment of the invention results where repeated cycles of freezing and thawing of polymer solution result in solids exclusion forcing polymer units in proximity with one another, probably through Van der Waals attractions, and possibly through ionic bonding, which leads to the generation of solid hydrogel. Preferably, the polymer is frozen at less than −20° C. for 10 to 20 hours, where cyclic freeze-thaws result in tighter bonding. Such a process generally does not result in degradation or denaturation of substrates within the polymer, and therefore is suitable for use with protein substrates.

The hydrogels utilize commonly occurring tissue and proteins, such as albumin, collagen, elastin, but may also contain silk, lignin, dextran, or may contain soy derivatives, polyglutamic acid. The biocompatible proteins preferably are elastin, albumin or collagen and are present at concentrations of about 1% to about 75% and more preferably 1-20%.

Additionally, pharmaceuticals, e.g., an anti-coagulant, an antithrombotic, an antibiotic, a hormone, a steroidal anti-inflammatory agent, a non-steroidal anti-inflammatory agent, an anti-viral agent or an anti-fungal agent, may be beneficially added to the hydrogel in order to provide some desirable pharmacologic event. In this further embodiment, a drug, biologic or other medicament may be absorbed into the matrix of the polymer resulting in a deposition composition.

Further, said hydrogel may be biomimetic, where chosen surfaces may be altered to suit desired tissue situation. Surface functionalization, surfactants, living cells, active functional groups may all be incorporated to aid in seal and active healing of hydrogel adhesive.

There are a number of surgical procedures involving implanting a biocompatible structure into the body whereby it provides support for an organ or other tissue. For example, in particular hernia operations, or in pelvic reconstruction, an inert mesh, sometimes made up of polytetrafluoroethylene or a biomaterial isolated from animal gut, is sutured into place with the intention of providing support, and improving with the infiltration of viable tissue. For these procedures, a polymer as described herein may provide support during hernia repair, or for pelvic reconstruction, for example, whereby the material is welded into place, with or without sutures. In this embodiment, the polymer may optionally have holes that improve the ingrowth of tissue.

Mechanical properties of said hydrogel such as flexibility, porosity, flow characteristics and rate of dissolution in liquid media are adjusted through concentration alterations in said polymer—typically, 1-25%. Also, these properties adjust through length or degree of crosslinking. Rates of biodegradation may also be manipulated by variations in these properties, and specific substitutions made to polymer increase or decrease degradation rates.

In all crosslinking methodologies, the hydrogel may be shaped by suitable molding techniques. Molds may be comprised of many materials that will not react with the polymer solution: glass, stainless steel, aluminum, brass, copper, Teflon, polystyrene among others. Polymer solution is poured into said mold prior to, during, or following crosslinking dependent on the crosslinking method: prior to, in the case of photopolymerization or lypholization; during, in the case of chemical crosslinking; following, in the case of thermal crosslinking. Preferably, the mold is Teflon and aluminum. More preferably, the mold is non-stick Teflon and non-stick aluminum. The molds may be cylindrical, annular, rectangular and so forth and are dependent upon physiological location of said hydrogel upon welding.

Other alterations made during or following crosslinking and molding can increase suitability of hydrogel for specific case. Size and thickness are determined through said molding techniques—typically, 1 mm-10 mm in thickness, more typically, 3-5 mm in thickness. To achieve desired thickness, hydrogel is subjected to extended pressure. Pressure varies from 1-20 pounds, more specifically, from 5-10 pounds. In addition, ideally, said hydrogel is manufactured so as to remove all air prior to crosslinking. Typically, vacuum or heat is used to release air from the polymer fluid.

There are other ways to form the thin-film polymer. For example, an extruder can push molten polymer through a die as a process (mechanical or vacuum) pulls the molten polymer out of the other end of the die; a process referred to as drawdown. In this case, the die will determine the thickness of the thin film. Another method involves dipping a substrated (such as the insert part of the patch) into a bath of the molten active polymer (such as the material with the susceptor and protein). Alternatively, one can take this material and draw it down to the appropriate thickness by placing a suitable volume on a spinning disk or on the inside of a rotating centrifuge drum.

The fusion composition may be composed largely of a protein, such as serum albumin, with the addition of ionic solution namely sodium chloride. The induced electrical currents produced in the salts results in heat which then conducts into the area immediately surrounding the metal, resulting in a “melting” of the hydrogel adhesive and perhaps the adjacent tissue. When the hydrogel adhesive cools, less than a second later, it forms a bond with the tissue, perhaps through cross-linking of the proteins. In tissue, we hypothesize that the temperatures needed to achieve a bond range from about 45-85° C., and the heating times are very short since protein denaturation is essentially instantaneous once a critical temperature is achieved.

The hydrogel and/or the conductive elements of the present invention may be used in methods of fusing, welding or creating a bond between tissues or between tissue(s) and another material such as, but not limited to, a tissue, a dressing, a fastener, or other biocompatible substrate. The hydrogel can be used as a sealing agent to seal a sinus in a tissue, to aid in forming an anastomosis between tissues or as an adhesive to adhere a dressing or other wound covering or fastening material to tissue(s). Furthermore, the conductive material itself may function as a fusion composition. The conductive material, e.g., metal particles, may be placed on or between the tissues or tissue(s) and other substrates to inductively form a weld or seal or bond.

The hydrogel and/or the conductive elements of the present invention may also be used in methods of filling tissue defects or for expanding tissues, for example, for cosmetic purposes.

Optionally, destabilizing/stabilizing agents, e.g. alcohol, can be added as they have been shown to alter the denaturation temperature. For example, an increase in the concentration of NaCl, referred to as “salting-in” proteins, can increase the denaturation temperature of lactoglobulin, while an increase in the concentration of NaCl0₄, or “salting-out”, reduces the denaturation temperature (9). When proteins are exposed to either liquid-air or liquid-liquid interfaces, denaturation can occur because the protein comes into contact with a hydrophobic environment. If allowed to remain at this interface for a period of time, proteins tend to unfold and to position hydrophobic groups in the hydrophobic layer while maintaining as much charge as possible in the aqueous layer. Thus, by ultrasonically adding bubbles to the composition will serve to lower the denaturation point of the mixture.

In the case of proteins, bioactive agents, and destabilizing/stabilizing agents, the materials can be forced into solution with the polymer prior to crosslinking or may be absorbed through diffusion into hydrogel following crosslinking.

A preferred embodiment of the invention is the presence of ions or metal as conductive elements in the composition which can be inductively heated as a result of the formation of eddy currents or its magnetic permeability, respectively. Optionally, the conductive elements may be present in a polymer layer separate from that which contains substrate. For example, a matrix consisting of two separate layers of hydrogel may have protein in one layer and salt in another so as to minimize the tendency for protein to “salt-out,” thus maximizing protein concentration in one layer.

Preferably, the polymer is inductively heated. However, where polymer materials are juxtaposed to tissues of low conductively, electric fields may be used to generate currents that flow through the conductive polymer medium, and capacitively couple with the low conductivity tissue.

The conductive materials that can be inductively or conductively heated are added to the polymer in amounts typically in concentrations of from 0.1 to 25%, more preferably 0.9-6%. Higher concentrations may be used under circumstances where effects of the conductive materials on living systems are not a factor, and where the effects on the crosslinking capabilities of the polymer are not a factor. The conductive material may be composed of salts or other ionic substances, or metals of variable size, depending on the operational frequencies. Additionally, the metallic materials may be an alloy with a Curie point in the range of about 42° C. to 99° C. Thus, when treating tissues and cells, a maximum temperature can be set according to the alloy. Generally, the range of useful particle sizes is from nanometer size to macroscopic size particles up to 1 mm wide. The particles may be, but not limited to, spheroid, elongate or flakes. Alternatively, the conductive material may take of the form of a fine mesh or film, such as available from Alfa Aesar Inc (Ward Hill, Mass.).

Example of materials that may be useful by themselves, or in alloys, in the present method and composition are tantalum, niobium, zirconium, titanium, platinum, LPhynox (an alloy of cobalt, chromium, iron, nickel, molybdenum), palladium/cobalt alloy, magnetite, nitinol, nitinol/titanium alloy, titanium (optionally alloyed with aluminum and vanadium at 6% Al and 4% V), tantalum, zirconium, aluminum oxide, nitonol (shape memory alloy), cobalt (optionally alloyed with chromium, molybdenum and nickel, or optionally 96% Co128% Crl6% Mo alloy), iron, nickel, gold, palladium, and stainless steel (optionally biocompatible type 316 L). The conductive materials may take the shape of a mesh, fibers, macroscoaic and solid materials, flakes or powder. The conductive materials may be anodized and may further be encapsulated in materials such as liposomes, compounds such as calcium phosphate, polystyrene microspheres, pharmaceuticals, or Teflon. The conductive materials may also be complexed with glass and ceramics, or embedded in epoxies or plastics. These complexes and encapsulating materials may minimize immune responses, or toxic reactions to the conductor, could induct a desirable pharmacologic event, or could enhance the inductive coupling to the activating magnetic field.

Upon being exposed to electromagnetic energy, or to the heat generated there from, the molecules in the material containing the electrically conductive element change in conformation, altering their interaction with each other or with molecules in the surrounding environment. For example, upon heating, protein or hydrogel may become more fluid, and flow into a second material, whereupon the molecules assume a different conformation upon cooling, thus enabling them to cross-link with molecules in the second material.

Alternatively, the electrical energy provided to the conductive element is provided by a battery incorporated into the polymer. Given that the temperature rise necessary to cause the beneficial thermal alterations are no more than 60° C., and more likely 30° C., the energy in the polymer can be low enough that only a very small battery is required.

The conducting element may also have geometry, e.g. a coiled configuration that efficiently inductively absorbs ambient radiofrequency energy. For example, a coil which is attached to a radiofrequency power-source external to and superimposed proximally will produce a magnetic field around the polymer. The conductive element is thus heated leading to thermal alterations of the polymer material which then effects a tissue-weld at the surface of the skin. The conductive element may also provide a means of measuring the heat generated in the system allowing for monitoring at a distal location. The conducting element may optionally be removed after the tissue fixation treatment, through physically withdrawing the element or through dissolving and absorption as a result of physiological processes. This may be accomplished, for example, through the use of conductive metals that are either solid or mixed in a polymer.

Provided herein are devices and methods for heating non-conventional substrates, i.e. polymer biomaterials, in order to cause adhesion. Preferably, the instant invention provides a device comprising a source of radiofrequency (RF) energy coupled to an applicator, which then produces an oscillating magnetic field, and the polymer which inductively couples with the magnetic field, resulting in the transient production of heat substantially within the composition. Inductive coupling most simply results in heating through dipole formation and hysteresis in particles or through eddy current formation where ionic species are impregnated in the biocompatible polymer adhesive. The device may create a weld or a bond between tissues or between tissue and some other material.

An embodiment of the present invention provides a method of heating biological materials comprising the steps of placing the device described supra proximate to the biological materials; and inductively heating said fusion composition comprising the device or a combination thereof to effect heating of the biological materials where the step optionally may be repeated at least once. This embodiment further may comprise the step of monitoring the device to control the extent of heating where the step optionally may be repeated at least once.

Another embodiment of the invention is a composition, a polymer, such that, upon inductive or conductive heating, the material undergoes a structural change which is detectable through a change in reflected power. This change is generally one that restrict motion of particles or ions within the polymer and includes coagulation and desiccation.

Control may be exerted by direct feedback monitoring of heat generation or by prediction and measurement of the magnetization of the composition over time with regard to its volume and mass. This feedback may arise from measurements of impedance changes in the applicator, as the tissue becomes part of the circuit during treatment, or devices such as thermocouples or infrared thermometers may be utilized. Another order of control may be exerted through the use of ferromagnetic metals and alloys as susceptors which remain magnetized until reaching a critical temperature, the Curie temperature, whereupon the cease to be magnetic.

Another embodiment of the present invention provides a device to heat biological materials comprising a fusion composition and a means to inductively generate heat to effect heating of the biological materials. The device may further comprise a means to control the extent of heating. Examples of such means is electronic, a means to monitor the thermal history of the device or a means to detect changes in a ferromagnetic material comprising said fusion composition. The biological material may be a tissue, a dressing or a fastener. The induction coil and the coating and components thereof are as described supra.

The power-supply is able to produce radiofrequency energy in the frequency range of of 100 kHz to 5.8 GHz, more preferably between 350-800 kHz, or at 869 MHz, 900 MHz, 2.4 GHz, 13.56 MHz, 27 MHz, 60 MHz, 81 MHz, or 5.8 GHz. The power in the range 1-5,000 W and may typically operate at frequencies of 100 kHz to 15 GHz. The power of the RF energy is in the range of 1-5000 W, and depending on the application, may be more preferably in the range of 100-600 W.

Above said coil configurations may be altered for use in particular tissues. Namely, the coil material may be surrounded by conductive material namely sapphire, mica, silica glass, Delrin®, or materials of the like to increase to create heat transfer from internally circulating coolant to superficial layers of tissue or hydrogel being treated. Alternatively, the said coil may be surrounded by a non-conductive material having the opposite affect on the tissue or hydrogel. Namely, polystyrene, aerogels, polyurethane, polyvinyl chloride and other like materials.

The application of the radiofrequency energy may be controlled by circuitry such as a battery and switch. Additionally, the induction means may also have a feedback control circuit to monitor voltage and conductance.

### EXAMPLES

### Hydrogel Fusion Composition 1: Hydroxypropyl Cellulose

Hydroxypropyl cellulose (10% and 15% solutions, wt/wt) was prepared in ultra-pure water with 2% or 4% NaCl. The formulations were heated to 35° C. to initiate crosslinking and then quickly removed from heat. Albumin (Bovine serum, or ovalbumin; Sigma-Aldrich, St. Louis, Mo.) (20%, wt/wt) was added to the final solution and the material layered into a polystyrene circular mold. The resultant material was a viscous hydrogel. For this particular hydrogel, thickness and shape were irrelevant as it is too viscous to control the shape.

The hydrogel fusion composition was spread onto a Teflon piece to a rectangular shape of 30×10 mm. The Teflon piece was placed supra said fusion device and energized for a period of 30 seconds. Evidence of denaturation and coagulation was ascertained visually as the hydrogel changed colors. This was confirmed by probing the material with a needle to show increased viscosity.

### Hydrogel Fusion Composition 2: Aliginate Hydrogel

Alginate, (3% or 5% solutions, wt/wt), was prepared in ultra-pure water with 2% or 4% NaCl and stirred until solubilization. Albumin (Bovine serum, or ovalbumin; Sigma-Aldrich, St. Louis, Mo.), (20% wt/wt) was added to the formulations. The formlations were poured into a polystyrene circular mold. The preparations were immersed in 10% calcium chloride solution, resulting in a thin, flexible, somewhat pourous hydrogel.

A rectangular shape (30×10 mm) was resected from the hydrogel fusion composition and placed on a Teflon piece. The Teflon piece with hydrogel was placed supra said fusion device and energized for a period of 30 seconds. Evidence of denaturation and coagulation was ascertained visually as the hydrogel changed colors. The hydrogel increased in porosity upon thermal activation evidence by water evaporation and spongelike appearance. The hydrogel did not harden upon heating which was confirmed by probing composition.

### Hydrogel Fusion Composition 3: Hyaluronic Acid Hydrogel

Hyaluronic acid (1% or 4%) was dissolved in 0.2M sodium hydroxide. Albumin was added (30% wt/wt), followed by divinyl sulfone (0.07 mL in 5 ml). The solution was poured into polystyrene mold and allowed to form a hydrogel.

A rectangular shape (30×10 mm) was resected from the hydrogel fusion composition and placed on a Teflon piece. The Teflon piece with hydrogel was placed supra said fusion device and energized for a period of 30 seconds. Evidence of denaturation and coagulation was ascertained visually as the hydrogel changed colors. The hydrogel drastically increased in stiffness and porousity.

### Hydrogel Fusion Composition 3: Polyvinyl Alcohol Hydrogel by Photopolymerization

A 10% wt/wt solution of polyvinyl alcohol in 4% wt/wt NaCl was prepared by rigorously mixing and heating the solution. Five milliliter samples were poured into polystyrene molds and subjected to UV light overnight. The samples were hydrated, and 2 g of dry albumin was allowed to soak into the PVA network post-crosslinking.

A rectangular shape (30×10 mm) was resected from the hydrogel fusion composition and placed on a Teflon piece. The Teflon piece with hydrogel was placed supra said fusion device and energized for a period of 30 seconds. Evidence of denaturation and coagulation was ascertained visually as the hydrogel changed colors. The hydrogel became extremely glassy and brittle upon heating.

### Hydrogel Fusion Composition 4: Polyvinyl Alcohol Hydrogel by Lyophylization

Initially, a PVA formulation containing 10% PVA (wt/wt), 4% NaCl (wt/wt) was mixed and heated at 95° C. until the components solubilized, then allowed to cool, and 20% albumin (wt/wt) added. The solution was poured into a polystyrene mold and frozen for 12 hours.

A rectangular shape (30×10 mm) was resected from the hydrogel fusion composition and placed on a Teflon piece. The Teflon piece with hydrogel was placed supra said fusion device and energized for a period of 30 seconds. Evidence of denaturation and coagulation was ascertained visually as the hydrogel changed colors. The hydrogel became increasingly flexible with the addition of heat, at the cessation of heat the hydrogel returned to a rubbery state.

The feasibility of the hydrogel was tested in vitro. A sheep lung ex vivo was placed on a countertop and standard surgical forceps were used to displace a small portion of tissue for stapling. The tissue section was clamped, stapled and cut using an Endo GIA 30 minimally invasive stapler (US Surgical), resulting in a staple line approximately 30×3.5 mm. Two strips of fusion composition 4 (each 25×300 mm) were placed juxtaposed to on either side of the staple line. The hydrogel was again subjected to 300 W of power for a period less than 30 seconds. It was apparent that denaturation had occurred and renaturation or crosslinking of proteins had also occurred by interrogating the sample with a needle probe. The hydrogel should adherence to tissue and no thermal damage to surrounding tissue.

### Hydrogel Fusion Composition 5: Polyvinyl Alcohol Laminate Hydrogel by Freeze-Thaw

A laminate composed of a single albumin-PVA layer and a second NaCl-PVA was prepared whereby one layer of 10% PVA (wt/wt), 6% NaCl (wt/wt) was mixed and heated at 95° C., allowed to cool, and then poured into a Teflon mold, 13×13×0.25 cm, lined with non-stick aluminum foil. The hydrogel was additionally covered with non-stick aluminum foil and subjected to one freeze-thaw cycle. A second preparation consisting of 10% PVA (wt/wt) was mixed and heated at 95° C. until components were dissolved. The mixture was allowed to cool to room temperature and then dry albumin was added to bring the solution to 17% wt/wt albumin, stirred until the albumin dissolved. The mixture was then added to the mold and again frozen overnight. The resultant polymer showed excellent tactile integrity and appeared to be elastic.

A rectangular shape (30×10 mm) was resected from the hydrogel fusion composition and placed on a Teflon piece. The Teflon piece with hydrogel was placed supra said fusion device and energized for a period of 30 seconds. Evidence of denaturation and coagulation was ascertained visually as the hydrogel changed colors. The hydrogel became increasingly flexible with the addition of heat, at the cessation of heat the hydrogel returned to a rubbery state. Additionally, the hydrogel showed evidence of flow upon heating.

The feasibility of the laminate hydrogel was tested in vitro. Intact sheep lungs were harvested from sheep. A lung was placed on a countertop and standard surgical forceps were used to displace a small portion of tissue for stapling. The tissue section was clamped, stapled and cut using an Endo GIA 30 minimally invasive stapler (US Surgical), resulting in a staple line approximately 30×3.5 mm. One strip of fusion composition (100×300 mm) was placed on the staple line. The hydrogel was subjected to 300 W of power for a period less than 30 seconds. It was apparent that denaturation had occurred and renaturation or crosslinking of proteins had also occurred by interrogating the sample with a needle probe. The hydrogel should adherence to tissue and little thermal damage to surrounding tissue.

The integrity of the seal was further measured through a burst strength test of the hydrogel adhesive staple line complex. The sample was loaded onto the Franz diffusion chamber, water was pumped into the chamber at a rate of 20 mL/min until the pressure was 22 mHg, and then maintained for 5 minutes to confirm integrity. The same procedure was followed till burst of the staple line. Six samples were completed in all and seals with hydrogel adhesive maintained average burst strength 30 mmHg higher than a staple alone. The hydrogel allowed for improved elasticity of the staple line and the flowability of the hydrogel increased adhesion at the wound site.

### Hydrogel Fusion Composition 6: Polyvinyl Alcohol Cylindrical Hydrogel by Freeze-Thaw

A cylindrical shaped hydrogel composed was prepared whereby 22% PVA (wt/wt) was mixed and 19.5% NaCl (wt/wt) was mixed. The PVA formulation was heated and stirred constantly until the polymer was dissolved in the aqueous solution. The two formulations were added together with the addition of albumin so that the end formulation was 40% albumin, 7.7% NaCl and 4% PVA. The formulation was then drawn into cylindrical molds 6 cm in length by 2 mm in diameter and frozen for 15 hours.

The feasibility of the hydrogel to seal sinuses in tissue was tested in vitro in porcine skin. The porcine skin was harvested and fatty and connective tissue removed. The tissue sample was cut to a square shape 5 cm×5 cm. At the center of the skin sample a wound was created with a 14G needle. The said hydrogel was injected into the wound site. The site was subjected to 300 W power for up to 60 seconds.

The integrity of the seal was measured through a burst strength test whereby the sample was loaded onto the Franz diffusion chamber; water was pumped into the chamber at a rate of 20 mL/min until burst of the sealed sinus. The average seal strength was 227 mmHg.

### Tissue Fusion to Stop Post-Hemodialysis Bleeding

Formulations were sought that would (1) not completely desiccate and therefore crosslink with tissue upon heating, (2) could be easily converted into a filament structure. To achieve this objective, a number of plasticizers were considered as an addition to the base formulation of water, albumin and salt; namely, chitosan, alginate, and polyvinyl alcohol (PVA). All formulations performed satisfactory under initial bursting strength tests; however, a formulation of 7.7% salt, 4% PVA, and 43% albumin performed the best while maintaining a soft filament structure. Polyvinyl Alcohol used in all formulations had a molecular weight M_{w}: 145,000, degree of hydrolysis: 99.0-99.8%, and a degree of polymerization: 3300.

For bursting strength measurements, samples of porcine skin and vessel from an unrelated IACUC approved study at Colorado State University were cut into 5 cm diameter pieces. The center of each piece was punctured with a 14G dialysis needle. The puncture was confirmed on bursting strength apparatus in RMBI's laboratory. Approximately 40 μL of filament was inserted into the sinus and cured in place at 60.1 MHz and 300 W. The samples were then retested for resistance to pressure in the same apparatus as above.

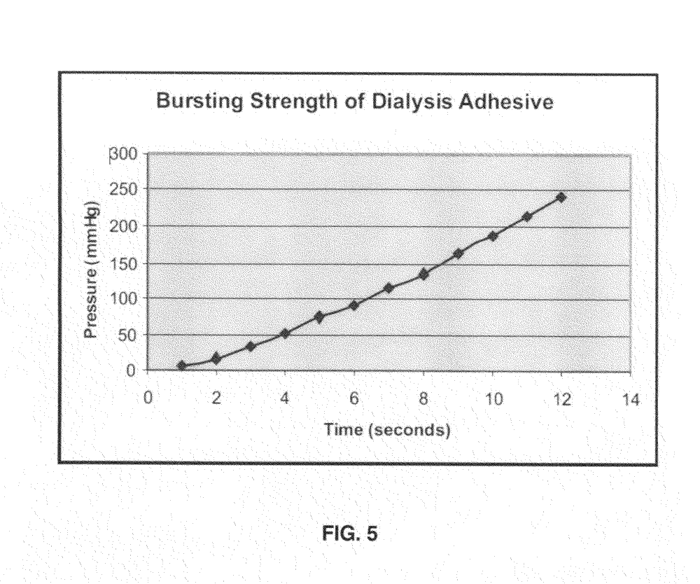

Prior to sealing, the tissue had an average resistance pressure of 8.5 mmHg with the sinus open (Table 1). With a seal, the strength increased to an average of 227.2 mmHg before rupture of the seal. The adhesive also showed consistent increased resistance to pressure with increasing pressure (FIG. 5) indicating a uniform seal had formed.

TABLE 1
Bursting strength of control (Pre-seal, punctured) and
sealed excised procine skin.

| | |
|---|---|
| PreSeal AVERAGE | 8.5 mmHg |
| PostSeal Average | 227.2 mmHg |
| MAX | 251.9 mmHg |
| MIN | 181 mmHg |

For formation of the filament, a freeze-thaw method was chosen instead of other polymerization methods that may result in excess protein cross-linking. Freeze thaw is an alternate cross-linking technique for PVA, whereby solids exclusion produces localized regions of attraction through van der Waals forces [i]. The freeze thaw process capitalizes on the optimal elasticity of PVA and controllable incorporation of proteins. For this step, 125 μL of the uncured adhesive was drawn into a Gilson (Middleton, Wis.) 125 μL pipette tip of the same diameter as the dialysis needle. The formulation and pipette were frozen at −20° C. for approximately 15 hours.

### In Vitro Tissue Evaluation

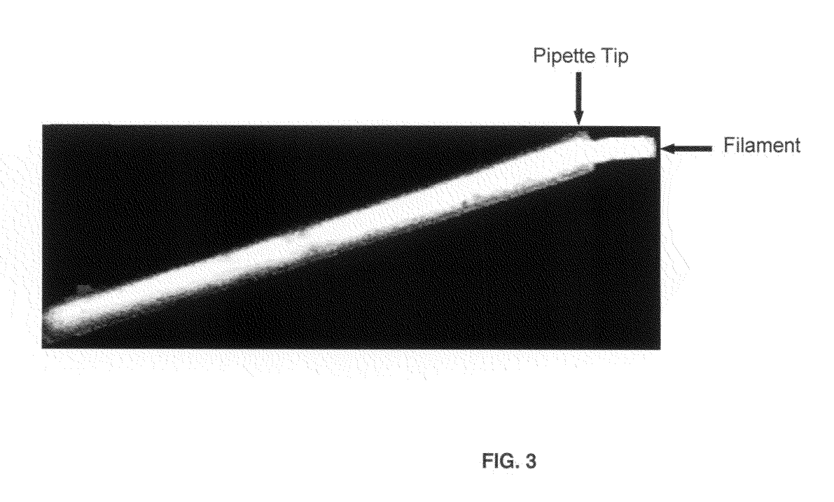

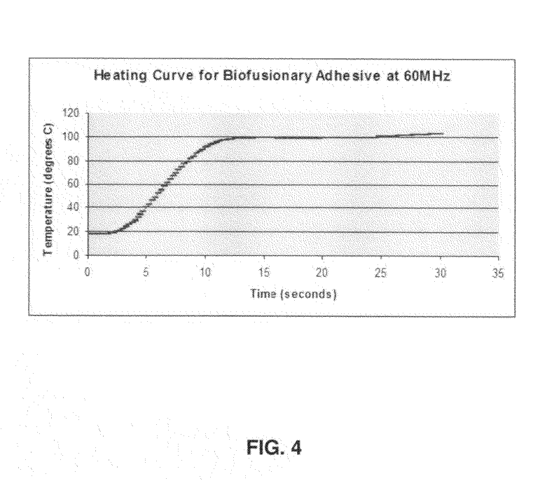

The resulting filament from (FIG. 3) was deployed into excised porcine skin sinus, and sealed in place to determine initial parameters for curing time, as well as to observe adverse tissue damage. These experiments demonstrated histologically that excess heat was generated at the surface of the skin, and the tissue damage was significant. For this reason, an advanced cooling module was developed that would cool the tissue surface, while permitting heating beneath the skin surface. Histology following initial experiments performed on skin samples indicated that the cooled faceplate prevented surface damage.

Another experiment was done to test a viscous form of adhesive, in the sinus created by a dialysis needle in live swine, and curing with either inductive or capacitive coupling. In this case, the sinus was created by a 14G tatoo needle which perforated full-thickness skin in the anesthetized swine. Then the adhesive (approximately 0.2 ml) was injected into the sinus with a positive-displacement pipettor. Immediately afterwards, the adhesive was cured by either application of 4 MHz RF energy which was applied with a Thermage Thermacool monopolar RF device using a 1 cm² tip, or with 40 MHz RF energy inductively coupled to the tissue via a pancake coil. In the capacitive situation, a ground electrode pad was necessarily affixed to the shoulder of the animal. Approximately 50 J of energy (capacitive coupling) was sufficient to seal the sinus and stop any bleeding that was present. Inductive treatment using approximately 200 W of 40 MHz RF power and treatment times varying from about 30-60 seconds also cured the adhesive. Upon gross examination of the capacitively coupled sites, little or no thermal damage was apparent on the superficial skin adjacent to the perforation site. All animals were examined for several hours after treatment, and then were observed daily for sequelae, but none were noted.

### Sealing Lobectomy Air Leaks by Inductive Coagulation

The Biofusionary adhesive was compounded as follows. Briefly, two layers of polyvinyl alcohol hydrogel were compounded, one layer containing salt and the other containing protein. The salt and protein serve as the active components of the adhesive and the polyvinyl alcohol creates a tissue like hydrogel for embedding the protein. Polyvinyl Alcohol has been shown to be biocompatible and potentially useful as a hydrogel in soft tissue replacement and drug delivery. [Cascone et al., Journal of Materials Science 10 (1999) 431-435; Cavalieri et al., Biomacromolecules 5(6):2439-2446, 2004] 2×2 cm samples of adhesive were placed on a microscopic slide positioned on a warming plate set to 37° C. The coil was fixed to a lab stand and positioned above the tests material. A Luxtron One fiberoptic thermometer (Santa Clara, Calif.) was positioned between the coil and the adhesive. The temperature was recorded at 4 Hz and the output was monitored by a microcomputer. Heating took place at 300 W (60 MHz), until the sample (ex-vivo lung tissue) temperature reached the cure point of 75° C. or for 30 seconds. The experiment was repeated a few times with great consistency (n=3). The device and Biofusionary adhesive worked well in tandem; the adhesive cured within 7 seconds.

### Pressure Testing Ex Vivo of Intact and Raw Lung Tissue Surfaces

Rabbit lung tissue was harvested at necropsy, dissected, with the parenchyma exposed and sealed with the THS (tape hydrogel sealant: (1) 50% Albumin, (2) 3% NaCl, (3) 1.5% HPMC, (4) 0.34% _-carageenan, (5) 1.5% gelatin dissolved in 9% gelatin solution) technology. Pressure testing will be done to confirm the quality of the seal. Gross examination and histology will be used to determine the extent with which the tissue participates in the sealing process and to which it is altered following treatment.

An objective was to develop a sealant which behaved similarly to lung tissue. An initial Tape Hydrogel Sealant (THS) was successful in sealing liver tissue; however, there was inconsistency between batches. If the Tape Hydrogel Sealant was allowed to dry too long, the formulation became brittle and inappropriate for a lung application. Also, the high amount of protein, though ideal for crosslinking, was a limiting factor in optimum biomechanics. It was hypothesized that a formulation with mechanics similar to lung tissue with strong crosslinking performance would outperform the Tape Hydrogel Sealant especially when considering the cyclic stress of inspiration and expiration of lung tissue. Also, patients undergoing lung surgery generally have very weak diseased tissue. Introducing an adhesive with stronger mechanics than the innate tissue could possibly introduce additional stress concentrations and translate to failure. Therefore, a formulation with strong elastic characteristics similar to lung would perform better. Polyvinyl Alcohol (PVA) was procured for its elasticity and biocompatibility. Numerous PVA formulations were tested before arriving at the current formulation. Sodium Tetra Borate (STB) was also procured as a known crosslinking agent for PVA—all chemicals were from Sigma Aldrich, St. Louis, Mo.

The first formulation was 3.5 w/w % PVA, 0.03 w/w % STB, 74 w/w% H2O, 11.4 w/w % albumin, & 4.5 w/w % NaCl. PVA used in the formulation had a molecular weight Mw: 145,000, Degree of Hydrolysis: 99.0-99.8%, and a degree of polymerization: 3300. PVA was heated and dissolved in H20, Albumin was added dry and salt was added in solution and mixed. STB was then added and the solution immediately crosslinked into a strong gel. The gel was then placed on nonstick aluminum foil and held under 15 lbs. of pressure until a flat piece of uniform thickness resulted. The formulation was then allowed to dehydrate for up to 24 hours to concentrate. Over 50% of water loss was recorded in 24 hours, nearly doubling the concentrations of albumin and NaCl. Samples were tested on tissue with 60 MHz and 300 W and showed good tactile crosslinking strength with the tissue. However, we observed the samples were porous. A formulation that is meant to be air tight could not be porous. The samples were also still inelastic when compared to the tissue. Therefore, crosslinking with STB was abandoned.

PVA crosslinking with ultraviolet was then considered for non-porous samples. PVA used in the formulation had a molecular weight Mw: 145,000, Degree of Hydrolysis: 99.0-99.8%, and a degree of polymerization: 3300. Briefly, a 10% wt/wt solution of polyvinyl alcohol in 4% wt/wt NaCl was prepared by rigorously mixing and heating the solution. Five milliliter samples were poured into Petri dishes and allowed to crosslink under UV light overnight. Note that addition of albumin prior to the UV cross-linking step results in denaturation, rendering the material unsuitable for use. Therefore, the samples were hydrated, and 2 g of dry albumin was allowed to soak into the PVA network post-cross-linking. Though the apparent superior elasticity, this formulation presented difficulties in controlling protein concentration.

Due to the uncontrollable protein absorption, another method of formulation, freeze-thaw, was utilized. Freeze thaw is a common cross-linking technique specific to PVA, whereby solids exclusion produces localized regions of attraction through van der Waals forces. Briefly, polymer undergoes periods of freezing and thawing to initiate the solid exclusion. The freeze thaw process capitalizes on the optimal elasticity of PVA and controllable incorporation of proteins. PVA used in the formulation had a molecular weight Mw: 145,000, Degree of Hydrolysis: 99.0-99.8%, and a degree of polymerization: 3300. Initially, a PVA formulation containing 10% wt/wt PVA, 4% wt/wt NaCl was mixed and heated at 95° C. until the components solubilized, and then allowed to cool, and 20% albumin (wt/wt) added. The solution was poured into a Petri dish and frozen for 12 hours. The resultant hydrogel contained clumps, suggesting salting out of protein, presumably due to the relatively high salt and PVA levels. This complication was addressed by mixing albumin or NaCl into separate PVA solutions, freeze thawing those solutions separately resulting in confluent hydrogels respectively. These separate hydrogels could be manufactured to be “layers” of one hydrogel; thus, the essential proteins and salts could be incorporated into one unit: each in their respective “layers”—a bi-layer formulation. Thus, the freeze-thaw formulation was prepared as a bi-layer structure, whereby one layer of 10% wt/wt PVA, 4% wt/wt NaCl was mixed and heated at 95° C., allowed to cool, and then subjected to one freezethaw cycle. A second preparation consisting of PVA 10% wt/wt was mixed and heated at 95° C. until components were dissolved. The mixture was allowed to cool to room temperature and then dry albumin was added to bring the solution to 17% wt/wt albumin, stirred until the albumin dissolved, adding 7.5 ml of this mixture to a Petri dish and freezing overnight. The resultant polymer demonstrated excellent tactile integrity and elasticity. However, the protein layer was not uniform in thickness and the presence of opaque regions suggested inconsistent distribution of the albumin.

A further modification to the production process was made to correct irregularities in the polymer. The PVA bi-layer formulation was subjected to molding under pressure to increase control. A Teflon mold, 13×13×0.25 cm, was created and lined with non-stick aluminum foil. The first freeze-thaw layer (PVA+NaCl) was poured into the mold, covered with a layer of non-stick aluminum foil and subjected to constant uniform pressure while freezing. The mold was then removed from the freezer, the polymer exposed, and the second freeze thaw layer (PVA+albumin) was poured on top. The polymer was covered with nonstick aluminum foil and subjected to constant pressure while freezing. The mold was removed from the freezer after 12 hours. The confluent, bi-layer, polymer that resulted from this process demonstrated excellent protein distribution, while maintaining elasticity and maneuverability. The polymer showed little porosity which was important for an air tight lung application.

PVAs of varying molecular weights were then tested in the bilayer formulation (PVA 56-98, 28-99, 10-98), see Table 2. Altering the MW would allow one to input additional proteins into the second layer of the bilayer and thus increase the adhesion potential. The above formulations were made with PVA 28-99. The same methodology was used to formulate the following samples; however, 17%, 25%, and 30% protein concentrations were all tested in the second layer of the bilayer. To compare adhesionm potential an ex vivo lung tissue test was used. Briefly, ovine lung tissue was harvested and cut into 5 mm diameter round samples with the parenchyma intact on one side. The center for the sample was punctured with a 14G needle creating a sinus for fluid flow. The tissue was clamped in a modified Franz diffusion cell and inflated at 20 mL/min. The sinus was confirmed in the testing apparatus by visually inspecting fluid leaking through the sinus. The puncture site was then covered with 1.5×1.5 cm samples cured in place at 60 MHz and 300 W.

TABLE 2
Comparison of molecular weights, degree of polymerization,
and degree of hydrolysis of the three polyvinyl alcohols studied.

| Polyvinyl | Molecular | Degree of | Degree of |
|---|---|---|---|
| Alcohol | Weight (MW) | Polymerization | Hydrolysis |
| 28-99 | 145000 | 33000 | 99-99.8 |
| 56-98 | 195000 | 4300 | 98-98.8 |
| 10-98 | 61000 | 1400 | 98-98.8 |

The formulation was tested for adhesion or resistance to pressure and fluid flow. The adhesion strength for each sample was recorded as the pressure at leak when fluid was visibly leaking through the edge of the adhesive. PVA 56-98 showed a higher adhesive strength for all formulations meaning a higher resistance to fluid pressure see Table 3. PVA 10-98 was unable to create the formulations most likely due to a lower molecular weight. The high molecular weight of 56-98, though ideal for adhesive strength was not as compliant as the 28-99 formulations and would most likely tear away from a respirating lung. Therefore, though 28-99 did not show as great adhesion, this formulation was used as it exhibited more appropriate mechanical properties. There was a decreased in the mechanical integrity as the protein concentration was increased with the formulation. Therefore, 17% was used to see if the lower amount of protein, though not showing as much as adhesive strength at 30%, would be enough to seal the tissue without compromising the excellent compliance.

TABLE 3
Average adhesion strength of different molecular weight
polyvinyl alcohols with different protein concentrations.

| | Protein | Av. Adhesion |
|---|---|---|
| PVA | [C] | Strength |
| 28-99 | 17 | 15 |
| 28-99 | 25 | 22.6 |
| 28-99 | 30 | 29.5 |
| 56-98 | 17 | 25 |
| 56-98 | 25 | 50.5 |
| 56-98 | 30 | 66.5 |
| 10-98 | 17 | NA |
| 10-98 | 25 | NA |
| 10-98 | 39 | NA |

Ovine lung tissue was harvested and the tissue was frozen until use. At the time of testing the tissue was defrosted and warmed in a bath to 37° C. The tissue was cut into sample approximately 5 cm in diameter and 1 cm thick with the parenchyma intact on one side. In the middle of the in tact parenchyma a 2 cm×1 cm piece was excised to mimic the raw tissue following a lobectomy. The absence of the parenchyma was confirmed by placing the test sample in the bursting strength apparatus. Briefly, a modified Franz diffusion cell is attached to manometer and Harvard Apparatus pumping 20 mL/min of fluid against the tissue. The tissue sample is clamped in the diffusion cell with the excised portion centered in the cell. Without adhesive, water began to leak through the excised portion of the tissue immediately after beginning the test (n=3, average pressure=5 mmHg).

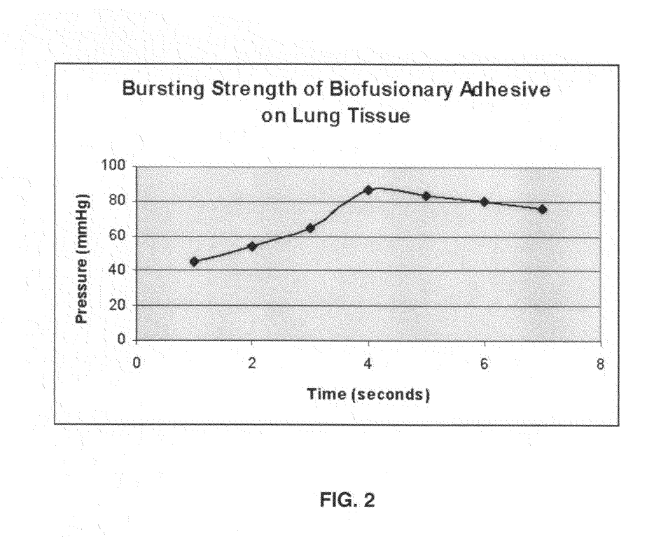

The sample was removed from the bursting strength apparatus and the wound created in the lung tissue was then covered with 3×3 cm piece of chosen adhesive. The adhesive was cured in place at 60 MHz and 300 W. The sample was placed back onto the bursting strength apparatus and tested as above until visible leak or burst (n=3, average pressure=51 mmHg, _{—}=14.9 mmHg; see FIG. 2). According to published values, a staple line in a lung volume reduction application will begin to leak around 30 mmHg (Downey et al. “Objective Assessment of staple line reinforcements.” Ann. Thoracic Surg. 82, 2006). This formulation withstood at least 40 mmHg at minimum, 68 mmHg at maximum.

### Defining Parameters the Adhesive of the Present Invention

The unique mechanical properties of tissue required a formulation that was not only biocompatible and cured in seconds, but also complimentary to the newly sealed tissue—mimicking its innate physical and mechanical properties. Therefore, loading the active components of protein and salts into a polymer hydrogel matrix would greatly increase the adhesives attractiveness and performance on tissue.

Alginate, hydroxyl propyl cellulose, PLGA, and PVA hydrogels were tested. After many mechanical, adhesion, and optimization studies, a bilayer PVA hydrogel was chosen which allowed a high concentration of salts in one layer and a high concentration of protein in the other. Additionally, the hydrogel could be prepared using a freeze thaw method that would avoid the denaturation of protein that occurs during ultraviolet and chemical crosslinking. The freeze-thaw process was conceived to develop tissue-like PVA hydrogels. PVA has been used in this form: Salubridge (Salumedica, Atlanta, Ga.) is an FDA approved nerve cuff composed of freeze-thaw prepared PVA hydrogel. Properties of the PVA bilayer adhesive are discussed herein.

For the modulus evaluation, a 30×10×5 mm sample of PVA was cut and placed between two rubber coated alligator-type clamps covered with sand paper to limit stress concentrations induced by the clamps. The modulus testing mechanism was confirmed with the known modulus of aluminum foil: on the order of 10⁹ Pascals. A digital video camcorder was positioned directly above the sample to collect images. The PVA sample was marked with a felt tip pen in four corners. These markings were used to analyze the change in dimension compared to the initial sample using the camera's frame grabber: sampling every 1/30th sec. The PVA sample was stretched at a rate of 0.5 mm/sec, and the load was recorded in kilograms. Prior to testing, the cross-sectional area was evaluated using digital calipers. This measurement was used as the area when deriving the stress at different time iterations for the sample (elastic modulus E=stress/strain; stress=force/area & strain=_L/L).

Elastic modulus for intact lung can vary as compared to excised lung tissue, a result of changes in load geometrics and perfusion of blood and air. Generally, the ex vivo lung tissue modulus varies from 10³ Pa to 10⁵ Pa. However, the modulus also varies due to location of the tissue on the lung itself, and often shows some degree of anisotropic behavior. The modulus of the chosen PVA formulation was slightly more elastic than published values for lung: 1.0×10⁶ Pascal. Intact ovine lung tested under the same conditions within the lab was 2.5×10⁵ Pascal (Table 4).

TABLE 4
Biomechanical comparisons of lung to PVA adhesive. PVA
bursting refers to staple-line reinforced with the PVA adhesive
(Hardness: n = 3; ±0.8%; Modulus: n = 3; s = 80 kPa;
Burst Strength: n = 6, s = 17.8 mmHg).

| | | Cured PVA |
|---|---|---|
| Property | Lung Tissue | Adhesive |
| Hardness (kg/cm2) | 1.0 | 0.78 |
| Modulus (Pa) | 2.5 × 10 5 | 1.0 × 10 6 |
| Burst Strength | 68.0 | 86.8 |
| (mmHg) | | |

Further analysis of batch to batch consistency was conducted by analyzing the tensile strength of the PVA hydrogel formulation. Tensile strength is a mechanical property that is affected by variations in chain entanglement. This measurement is used to demonstrate that the degree and nature of chain entanglement is consistent between different batches of the bilayer PVA hydrogel formulations.

Three samples of bilayer PVA hydrogel formulation from three different batches (n=9) were tested for tensile strength. For these evaluations, 15×35×20 mm samples of adhesive were clamped at each end and pulled apart at a linear rate of 1 cm/minute. Simultaneous measurements of force (in Newtons) were recorded using a digital force transducer (Ametek Accuforce). Tensile strength was calculated to be the maximum force divided by the cross-sectional area perpendicular to the force vector. Each of the nine samples had an average maximum force of 1.1 N (_{—}=0.1) and an average tensile strength of 0.47 Pascals (_{—}=0.13). With nine samples, the results produced outstanding consistency with a 95% confidence interval of 0.3935 to 0.05645 Pascals—a separation of only 0.17 Pascals.

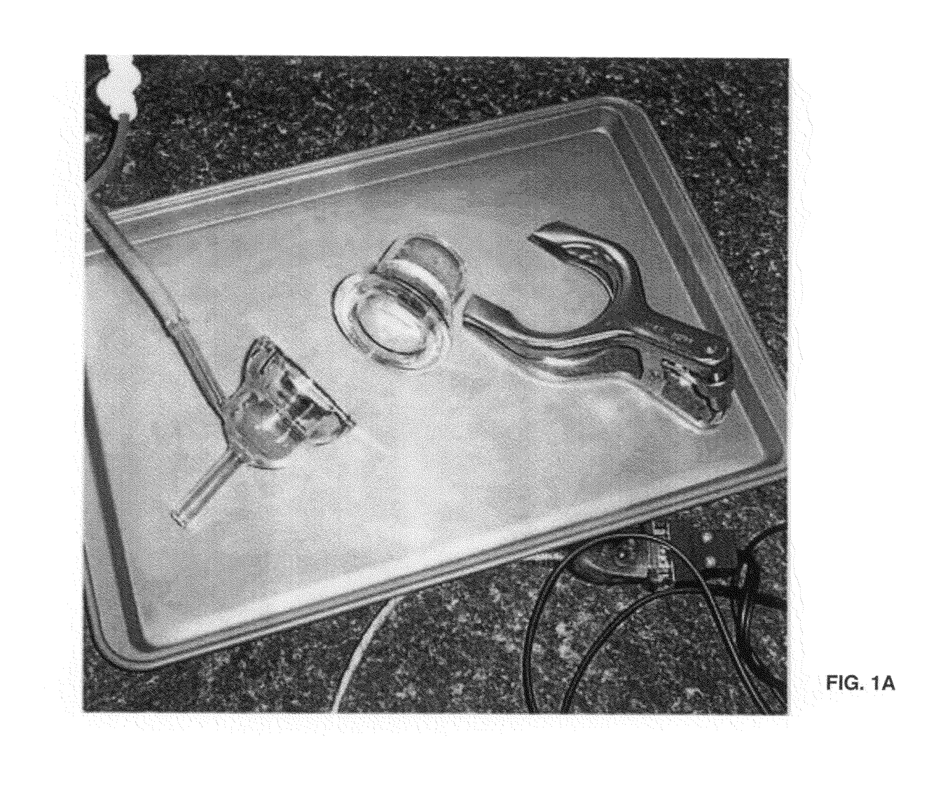

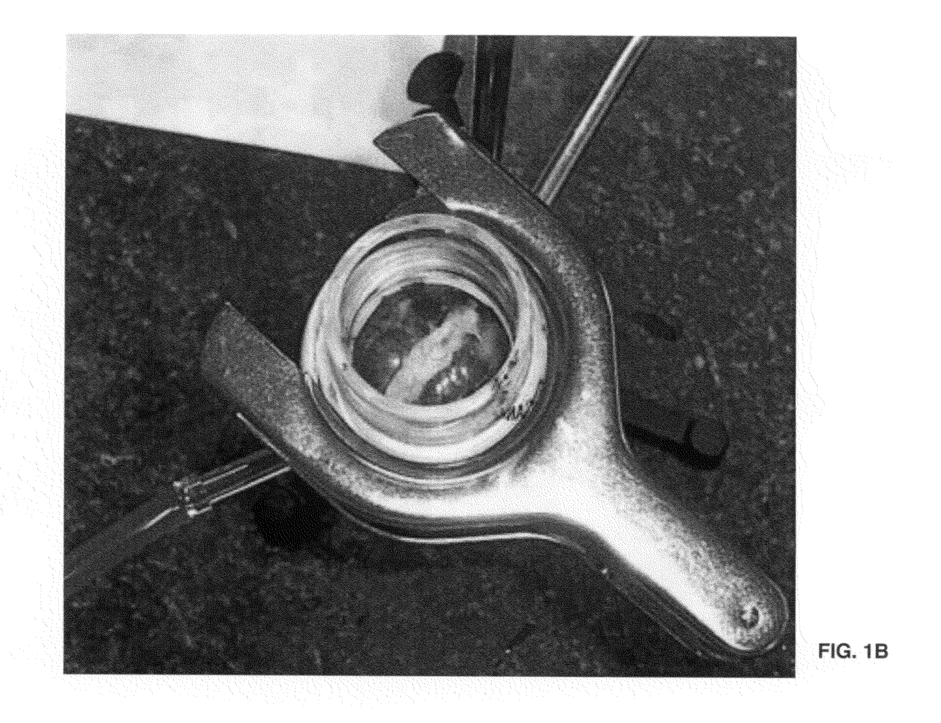

Additional tests of the bilayer PVA adhesive formulation were conducted in vitro by analyzing the burst strength integrity of the seal over a 3 mm staple line on ovine lung. Intact sheep lungs were harvested from sheep. Each lung was placed on a countertop and standard surgical forceps were used to displace a small portion of tissue for stapling. The tissue section was clamped, stapled and cut using an Endo GIA 30 minimally invasive stapler (US Surgical), resulting in a staple line approximately 30×3.5 mm. The treated portion of the lung was resected from the intact lung and was placed in a modified Franz diffusion chamber (FIG. 1A-1B). The apparatus was sealed and pressurized to 22 mmHg (static lung pressure) with water to identify leaks and confirm that there were no iatrogenic perforations or flaws within the tissue. The staple line was then covered with a 50×15 mm (approximate) piece of bilayer PVA adhesive. The bilayer PVA adhesive was cured in place using the Biofusionary handpiece applicator (60 MHz coil) held at a distance of 5 mm-10 mm from the tissue to produce an alternating magnetic field. The adhesive was exposed to the field until visual evidence of curing was apparent, but with a maximum cure time of no more than 30 seconds.

Following curing, the sample was again loaded onto the Franz diffusion chamber for further pressure testing. Water was pumped into the chamber at a rate of 20 mL/min until the pressure was 22 mmHg, and then maintained for 5 minutes to confirm integrity in the sealed staple line and tissue. The pressure was monitored and recorded throughout this step. Following the pressure test, each sample of tissue was subjected to increasing pressures in order to determine whether or not there were changes in the bursting strength of the tissue as a result of using the PVA bilayer adhesive to buttress the staple line.

Lung tissue with staples withstood an average maximum pressure of 42 mmHg, and the maximum pressure of any tissue sample was 70 mmHg. This average compares with published data on burst pressure that indicates non-reinforced staple lines leaked at 37 mmHg. Further research produced similar results: a non-reinforced staple line leaked at 30 mmHg and burst at approximately 60 mmHg.

In the tissue sealed with the formulation of the present invention, all samples withstood pressures greater than 70 mmHg. The average pressure withstood by bilayer PVA adhesive reinforced tissue in studies was 86.8 mmHg and the maximum pressure was 150 mmHg (see Table 5). These results exceed recently published results which indicate that staple lines reinforced with PTFE withstand 34 mmHg, and a staple line reinforced with bovine pericardium withstands 47 mmHg. In addition, the yield strength or the point where the “staple only” tissue begins to leak, is the same as the bursting strength or final failure for the staple line which is indicative of the adhesive's integrity. On the other hand, the staple line alone, leaked at half the pressure of its final burst, indicating that air leaks are likely to occur at 30 mmHg.

TABLE 5
Pressure tests of stapled and sealed/stapled lines in vitro ovine lung
(n = 5, s = 17.8 mmHg).

| | Staple | Staple Line with |
|---|---|---|
| | Line | Biofusionary |
| Min. burst pressure | 30 mmHg | 70 mmHg |
| Strength at Yield | 50 mmHg | 87 mmHg |
| Average Burst Strength | 68 mmHg | 87 mmHg |
| Max. pressure withstood | 69 mmHg | 150 mmHg |

Swelling characteristics of a hydrogel are critical in biomedical applications. The degree of swelling affects diffusion, surface characteristics, mechanical properties and surface mobility. Cavalieri et al., Biomacromolecules 5(6):2439-2446, 2004. Swelling can also reflect the degree of cross-linking of different hydrogel and polymer networks—increased space between network chains represents a lower degree of cross-linking and an susceptibility to volume increases upon addition of a solvent. The degree of cross-linking is directly related to the materials likelihood to biodegrade.

Swelling was evaluated in six different preparations of the PVA bilayer formulation of the present invention (total n=6 samples, FIG. 6). Briefly, 2×2 cm pieces of polymer were weighed and then immersed in 5 ml of water at 23° C. For each polymer sample, the mass swell ratio was determined each minute for thirty minutes. The mass swelling ratio quantifies swelling characteristics and is the ratio of swollen weight to dry or initial weight taken over time increments until equilibrium. The bilayer swelled quickly; reaching equilibrium at 200% within five minutes. Rapid swelling is suggestive of loose chain entanglement rather than cross-linking.

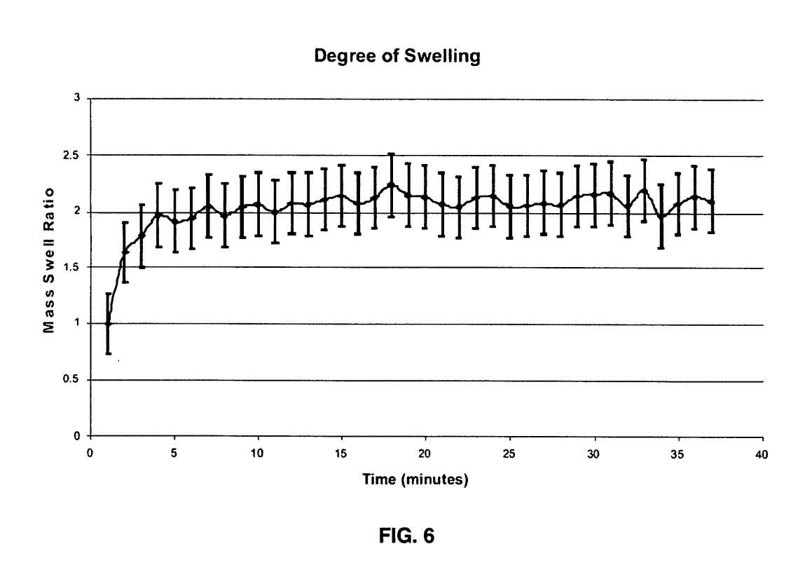

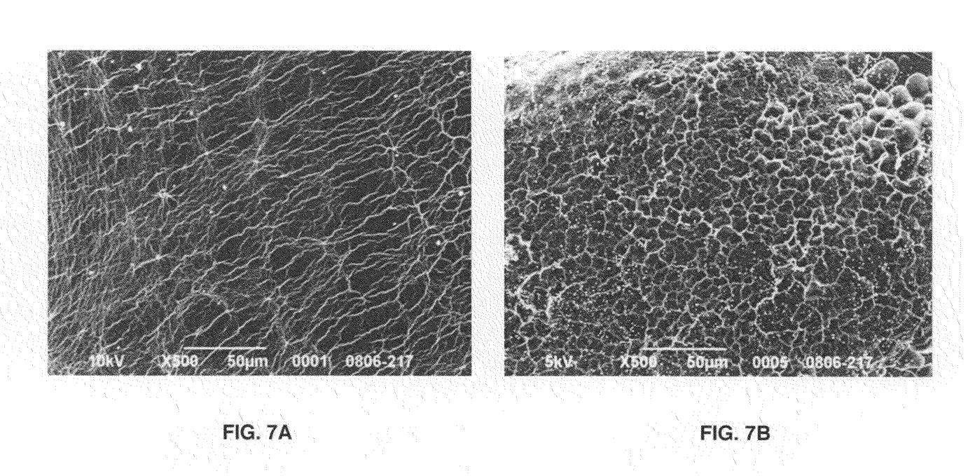

To establish an understanding of the morphology of the bilayer adhesive, an image of the bilayer polymer was captured with a Scanning Electron Microscope at Microbac Laboratories Hauser Division (Boulder, Colo.). The two layers of the bilayer were imaged separately. The first layer, the PVA and salt layer, was imaged at 500× and 10 kV and appears on the left in FIGS. 7A. The second layer, the protein layer, was imaged at 500× and 5 kV and appears on the right in FIG. 7B. Higher scanning powers tended to heat up the protein beyond 60° C. and thus rendered an inconclusive image. The PVA and polymer side shows presumed chain entanglement with a pore size visually estimated at 10-50 μm. The PVA and protein side does not show the chain entanglement of the first side, but rather evenly sized consistent pores of 10 μm.

### Adhesive Tissue Testing

After parameters of the bilayer polymer were defined. In vivo experimentation was completed to affirm the polymer's superior performance for the target application: sealing over staple lines following lung resections.

For the studies, six New Zealand White rabbits were used. Rabbits were intubated and anesthetized with a mixture of ketamine and xylazine. During the study, five animals had unsuccessful intubations and, therefore, received a tracheotomy. All rabbits underwent a thoracotomy with two to three ribs resected around the 5^{th} intercostal space. Following the thoracotomy, the right pulmonary lobes were exposed. In the first two rabbits, the right central lobe was stapled and resected, while the right cranial lobe was resected in the remaining four animals. An Endo GIA 30 minimally invasive stapler was used to staple the lobe, creating a staple line of approximately 3.0 cm in length. In order to maintain normal respiration, the lungs were continually ventilated at physiologic pressures (25 cmH₂₀ (18.4 mmHg)) as the thoracic cavity was filled with saline, and the staple line interrogated for air leaks for 5 minutes.

After the initial 5 minute ventilation period, a 3.0×2.0 cm section of bilayer PVA adhesive was applied to the staple line and cured along its length using non-overlapping 30 second exposures to the alternating magnetic field generated from the handpiece applicator, which operated at 60 MHz and 300 W power. Ventilation continued at 25 cmH₂₀ for 5 minutes, and the tissue was interrogated for leaks. Upon completion of the two ventilation procedures, lungs were hyperventilated to 40 cmH₂₀ (29.4 mmHg) or 60 cmH₂₀ (44 mmHg) and again allowed to respirate at the elevated pressure for 5 minutes.

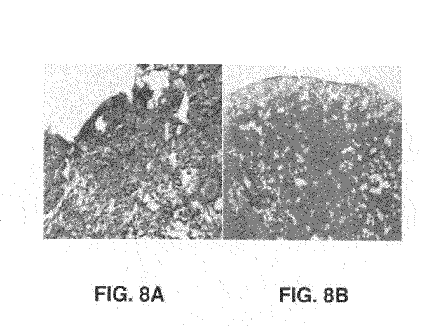

Air leaks were immediately observed in 50% of unsealed staple lines (Table 6). No leaks were noted post-curing, even during hyperventilation. Tissue samples from three of the stapled and sealed resected lungs were submitted for histology. In summary, lung tissue from all three samples appeared intact, viable and generally normal. A representative sample from one rabbit (FIG. 8A) appears as almost entirely healthy, normal lung, with minimal hemorrhage that may be procedure-related. A sample from a second rabbit (FIG. 8B) does show an area of fluid accumulation (edema), measuring 0.6 cm, with minimal inflammation and organization, within the center of the lung sample. Notably, the edema does not approach the pleural surface where the sealant was applied. A sample from a third rabbit showed a small amount of alveolar fluid near the pleura, but again appeared to be healthy overall. None of the samples showed evidence of giant cells, granulomas, scarring, fibrosis or any tissue necrosis.

TABLE 6
Ventilation tests of stapled and sealed/stapled lines in vivo
(n = 6, 40 cmH₂0; n = 3; 60 cmH₂0; n = 3).
All sealed lungs performed above 95% confidence.

| Treatment | | % |
|---|---|---|
| Condition | Pressure | Leakage |
| Staples alone | 25 cmH₂0 | 50 |
| Staples plus BF | 25 cmH₂0 | 0 |
| Staples plus BF | 40 cmH₂0 | 0 |
| Staples plus BF | 60 cmH₂0 | 0 |

Generally, the sealing studies depended on formulations that were composed, in part, of bovine or egg albumin. Ultimately, human albumin may be necessitated in clinical formulations to prevent tissue reactions and limit potential contamination with animal viruses. In order to demonstrate consistent performance with different protein sources, three different formulations of polymer were prepared. They included Human Albumin (Lee Biosolutions, St. Louis, Mo.), Bovine Albumin or Egg Albumin (Sigma Aldrich, St. Louis, Mo.). All albumins were formulated into the 2^{nd} layer of the polymer.

Following complete preparation and freeze-thaw cycles, the polymers were tested for adhesion to ovine lung tissue. Six centimeter diameter samples were excised to include intact parenchyma on one side of the sample and raw lung tissue on the other. The sample was then punctured in the center with a 14G needle and the sinus confirmed in a bursting strength apparatus. The sinus was then covered with a 1.5×1.5 cm sample of fusionary formulation of the present invention and cured in place at 60 MHz and 300 W. Following curing the samples were retested in the bursting strength apparatus and the pressure at which visible leakage was evident was recorded (n=3 samples for each protein formulation). All three albumins performed with equivalence (p<0.05) and had an average adhesion strength of 100 mmHg (Table 7).

TABLE 7
Adhesion strength of Human, Bovine, and Egg albumin is
equivalent (p < .05).

| | Adhesion Strength |
|---|---|
| Albumin | (mmHg) |
| Human | 104.3 |
| Bovine | 101.3 |
| Egg | 100.3 |

### Micro-Particle Biofusionary Formulations

The bilayer adhesive has displayed excellent performance characteristics as a lung tissue sealant; however, the adhesive is in the form of a film which may be difficult to deploy during a thoracoscopy. An adhesive that can be aerosolized, could then be deployed by spraying the formula onto the wound (the staple line). To determine feasibility of generating an aerosolizable form, three techniques for creating micro-particles based on the bilayer formulation of the present invention were tested for initial manufacturability and for adhesion to tissue.

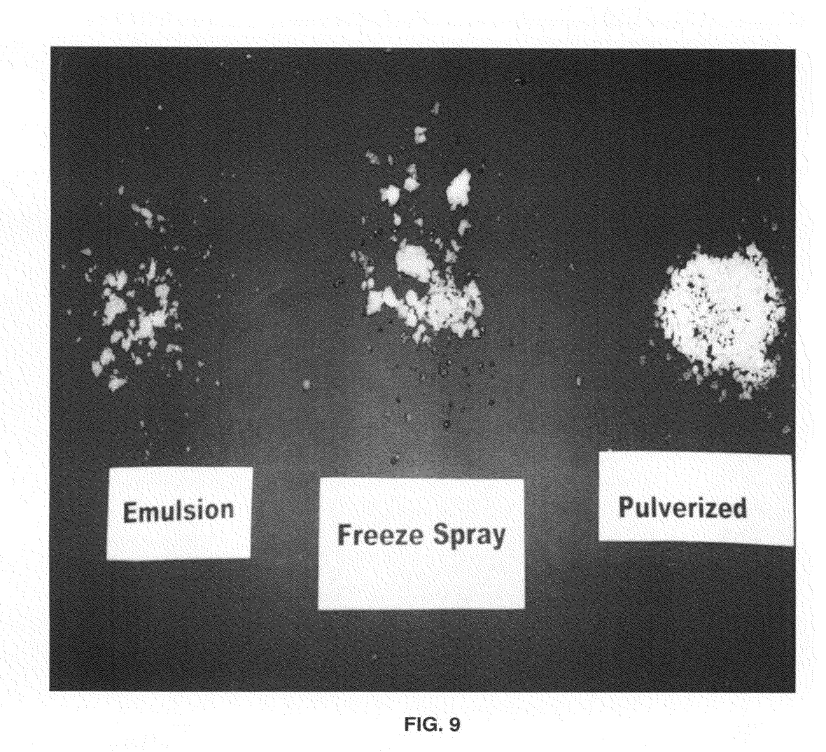

First, a water-in-oil emulsion technique was tested. A PVA solution (10% PVA, 6% NaCl, and 17% Albumin—the same concentrations as the bilayer adhesive) was first prepared. The solution was suspended in mineral oil (freezing temperature of −30° C.) and mixed at 3000 rpm for 30 minutes. The solution was then covered and frozen overnight. Two freeze thaw cycles were completed before an acetone wash (1:10 v/v) was used to remove the mineral oil and collect the PVA particles. The low viscosity of the mineral oil in comparison to silicone oil allowed the particles to migrate and aggregate during freezing. Additionally, the acetone wash denatured the protein and caused the proteins to aggregate, making the particles unproductive in an adhesion test. The method did produce a consistent sized particle (FIG. 9); therefore, alterations to the technique have been included below.

A second formulation was created by modifying the rapid freeze spray protocol of Barron et al. A solution of PVA was loaded into a Preval Spray Gun (Precision Valve Corporation) and the nozzle of the spray gun was aimed into a Dewar flask full of liquid Nitrogen at −196° C. The solution of PVA was initially 10% PVA, 6% NaCl, and 17% Albumin; however, the solution was diluted with 55 mL of Deionized water to make the solution sprayable. Micro-particles were created immediately when the PVA solution collided with the liquid Nitrogen. Once a layer of micro-particles covered the surface area of the liquid Nitrogen, the particles were removed and the process was continued. The final layer was left in the flask and the Nitrogen was allowed to evaporate. The micro-particles were then returned to the Dewar flask and stored in a −80° C. freezer until the adhesion test. Upon thawing, these micro-particles assumed a hydrogel form, and particles were less distinguishable, possibly due to incomplete polymerization as a result of rapid freezing. An additional freeze-thaw step (−20° C.) was then added to the protocol to ensure that the particles remained intact.

The final formulation tested was a pulverized bilayer, whereby, a bilayer adhesive was created and then pulverized by first chopping with a razor blade and then freezing it again inside a mortar at −80° C. for one hour. The mortar was then removed with the frozen polymer inside and a pestal was used to grind the polymer into a fine powder (FIG. 9).

An adhesion test was completed on two lung samples for both the rapid freeze spray and the pulverized formulations. Four tissue samples were excised from intact ovine lung tissue. The tissue was punctured in the middle with a 14G needle creating a sinus and the sinus was covered with a 2×2 cm area of micro-particles. The micro-particles were then inductively heated at 60 MHz and 300 W. As the particles heated, the hydrogel coalesced and flowed into the sinus. The adhesion was then tested by placing the tissue into a modified Franz diffusion cell and clamping it on all sides. The pressure was gradually increased until failure of the adhesive.

Both the pulverized formulation and the rapid freeze spray formulation maintained pressures up to 22 mmHg which is static lung pressure. Further modifications to the technique are included in the proposed research below to increase the overall adhesive properties. Namely, protein concentration will be increased to promote greater binding.

What is claimed:

1. An adhesive composition comprised of one or more layers of polymer, each of which may be comprised of a substrate and a conductive element.

2. The adhesive composition of claim 1, wherein the conductive element is an ionic salt, conductive polymers, transition metal, ionic solution, circuit pattern, or ferromagnetic material.

3. The adhesive composition of claim 1, wherein said substrate cross-links, welds, or fuses to tissue upon heat activation.

4. The adhesive composition of claim 3, wherein said cross-linking is achieved through chemical, thermal, lyophilization, freeze-thaw, or photopolymerization methods.

5. The adhesive composition of claim 3, wherein said heat activation is by inductive coupling or capacitive coupling.

6. The adhesive composition of claim 1, wherein said polymer is polyethylene glycol, polyvinyl alcohol, polylactic acid, polyglycolic acid, polycaprolactone, poly lactic-co glycolic acid, polyvinyl chloride, poly(vinylpyrro1idone), polyacrylamide, methacrylates, polyethylene oxide, polysaccharide, or glycosaminoglycan.

7. The adhesive composition of claim 6, wherein said polysaccharide is cellulose, hydroxymethyl propyl cellulose, aliginates, carageenans, or pullulan.

8. The adhesive composition of 6, wherein said glycosaminoglycan is hyaluronic acid.

9. The adhesive composition of claim 1, wherein said polymer is solid.

10. The adhesive composition of claim 9, wherein said polymer contains indentations or holes that allow tissues to grow into the polymer.

11. The adhesive composition of claim 1, wherein said polymer has been casted, molded or extruded to a specific three-dimensional geometric shape.

12. The adhesive composition of claim 11, wherein said geometric shape includes rectangles, tubes, cylinders, or cones.

13. An adhesive composition comprised of one or more layers of a biopolymer or a hydrogel, each of which may be comprised of a substrate and a conductive element.

14. The adhesive composition in claim 13, wherein said biopolymer is albumin, collagen, fibrin or elastin.

15. The adhesive composition of claim 14, wherein the elastin, albumin or collagen are present at concentrations of about 1-75%.

16. The adhesive composition of claim 15, wherein the elastin, albumin or collagen are present in concentrations of about 1-20%.

17. The adhesive composition of claim 13, wherein said hydrogel contains albumin, collagen, elastin, silk, lignin, dextran, soy derivatives, or polyglutamic acid.

18. The adhesive composition of claim 13, wherein said hydrogel contains a pharmaceutical, anti-coagulant, antithrombotic, antibiotic, hormone, anti-inflammatory agent, anti-viral agent, or anti-fungal agent.

19. The adhesive composition of claim 13, wherein the surface of said hydrogel may be functionalized or altered.

20. The adhesive composition of claim 13, wherein the conductive element is an ionic salt, conductive polymers, transition metal, ionic solution, circuit pattern, or ferromagnetic material.

21. The adhesive composition of claim 13, wherein the conductive element is separate but proximal to the hydrogel.

22. The adhesive composition in claim 13, wherein the polymer is substituted with a bifunctional or heterofunctional cross-linker.

23. The adhesive composition of claim 22, wherein said bifunctional cross-linkers include chains of polyethylene glcyol or polyethylene oxide substituted with end units such as hydroxyls, maleimids, sulfhydryls, esters, or amines.

24. A method for filling defects in tissue comprised of:
applying the polymer of claim 1 and exposing the polymer to a high frequency magnetic field.

25. A method for attaching tissue together, comprised of:
applying the polymer of claim 1 between two tissues, and exposing the polymer to a high frequency magnetic field.
